(19) United States
(12) Patent Application Publication
(10) Pub. No.: US 2011/0160585 A1
(43) Pub. Date: Jun. 30, 2011

(54) ULTRASOUND FOR NAVIGATION THROUGH PSOAS MUSCLE

(75) Inventors: Ephraim Akyuz, Providence, UT (US); Michael Wentz, Zionsville, UT (US); Daniel E. Gerbec, Logan, UT (US)

(73) Assignee: MEDICINELODGE, INC. DBA IMDS CO-INNOVATION, Logan, UT (US)

(21) Appl. No.: 12/827,921

(22) Filed: Jun. 30, 2010

Related U.S. Application Data
(60) Provisional application No. 61/222,187, filed on Jul. 1, 2009.

(51) Int. Cl. A61B 8/14; A61B 19/00

(52) U.S. Cl. 600/443; 128/898

(57) ABSTRACT

Imaging technology may be used to navigate highly innervated tissue, such as the psoas muscle, while maintaining the neural structures intact. An ultrasound transducer may be introduced into the tissue and an image may be consulted to assess the proximity of the transducer to neural structures. Alternate embodiments contemplate an expanded array of surgical applications and alternate imaging technologies.

### CROSS-REFERENCE TO RELATED APPLICATIONS

This application claims the benefit of U.S. Application No. 61/222,187, filed Jul. 1, 2009, entitled ULTRASOUND FOR NAVIGATION THROUGH PSOAS MUSCLE, Attorney's docket no. MLI-76 PROV, which is pending.

The above-referenced document is hereby incorporated by reference in its entirety.

### BACKGROUND OF THE INVENTION

Open surgical procedures afford surgeons a direct view of the surgical site and the opportunity to manually dissect superficial body structures covering the surgical site, so as to do minimal collateral damage to those structures. However, as minimally invasive surgical procedures become more popular, issues of surgical access and visualization become more critical. There is a need for apparatus and methods that enable surgeons to safely navigate through body structures to a surgical destination along a route at least partially defined by clinically relevant tissues in the vicinity of the route.

By way of example, surgeons may navigate through muscle while avoiding damage to nerves by using apparatus and methods that electrically stimulate nerves. However, these apparatus and methods do not provide a visual representation of the nerves or muscle, only a proximity warning similar to the “Hot” and “Cold” warnings used in the familiar childhood game. There remains a need for apparatus and methods that let surgeons see where to place their surgical access route.

### BRIEF DESCRIPTION OF THE DRAWINGS

Various embodiments of the present invention will now be discussed with reference to the appended drawings. It is appreciated that these drawings depict only exemplary embodiments of the invention and are therefore not to be considered limiting of its scope.

FIG. 1 is a cephalad view of an intervertebral disc, a vertebra, a psoas muscle, and a straight probe, with the psoas muscle shown in cross section;

FIG. 2 is a cephalo-lateral perspective view of an intervertebral disc, a vertebra, a psoas muscle, and a curved probe, showing a portion of the psoas muscle adjacent to the vertebral structures;

FIG. 3 is a transverse section view of a torso, showing a vertebra surrounded by muscles, nerves, blood vessels, and other organs;

FIG. 4 is a frontal view of a torso, showing the psoas muscle and nerves of the lumbar plexus.

FIG. 5 is a flow diagram of an exemplary method according to the present invention;

FIG. 6 is a flow diagram of an alternate method according to the present invention; and

FIG. 7 is a flow diagram of another alternate method according to the present invention.

### DETAILED DESCRIPTION

Standard medical planes of reference and descriptive terminology are employed in this specification. A sagittal plane divides a body into right and left portions. A mid-sagittal plane divides the body into equal right and left halves. A coronal plane divides a body into anterior and posterior portions. A transverse plane divides a body into superior and inferior portions. Anterior means toward the front of the body. Posterior means toward the back of the body. Cephalad means toward the head. Caudal means toward the feet. Medial means toward the midline of the body. Lateral means away from the midline of the body. Axial means toward a central axis of the body. Abaxial means away from a central axis of the body.

In this specification, “body” means the physical substance of an animal. “Flesh” means the soft parts of the body of an animal. “Tissue” means an aggregate of cells, usually of a particular kind, together with their intercellular substance, such as connective tissue, epithelium, muscle tissue, and nerve tissue. “Body part” refers to any part of an organism such as an organ or extremity.

In this specification, “clinically relevant” means having significant and demonstrable bearing on observable or diagnosable symptoms.

In this specification, “color” means all variations within the visible spectrum including white and black. “Hue” means the attribute of a color produced by a single wavelength of visible light that permits it to be classed as red, yellow, blue, or any intermediate value. “Tint” means a hue altered by the addition of white. “Shade” means a hue altered by the addition of black. “Tone” means a hue altered by the addition of grey.

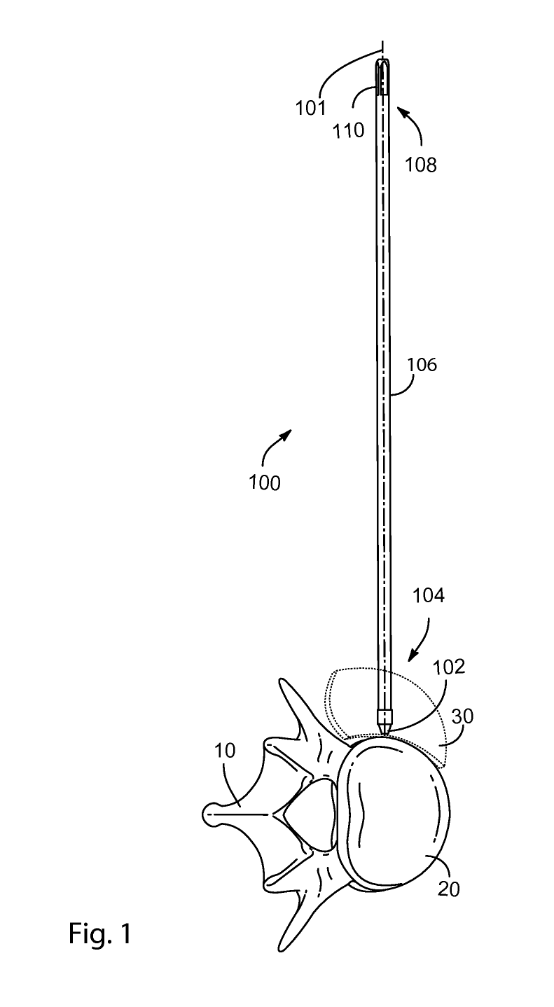

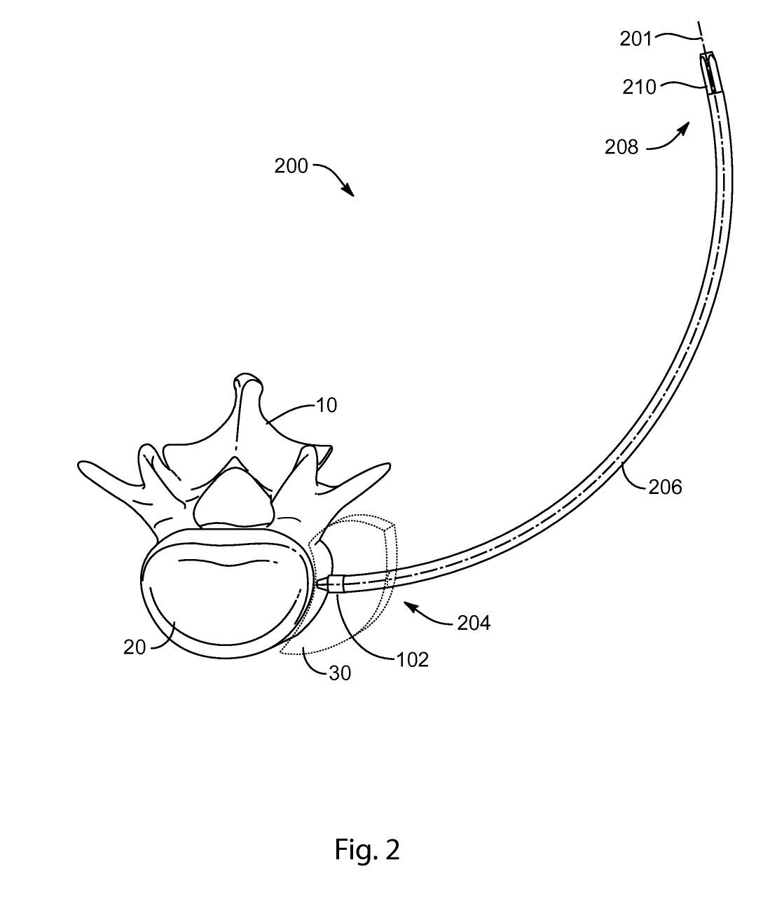

Referring to FIGS. 1-2, exemplary embodiments of an apparatus according to the invention will be shown and described in detail in the context of a lateral approach spinal intervertebral fusion procedure. While these embodiments are shown and described in the context of a specific surgical application, one of ordinary skill in the art will appreciate that the present invention has utility in other surgical applications in various parts of the body.

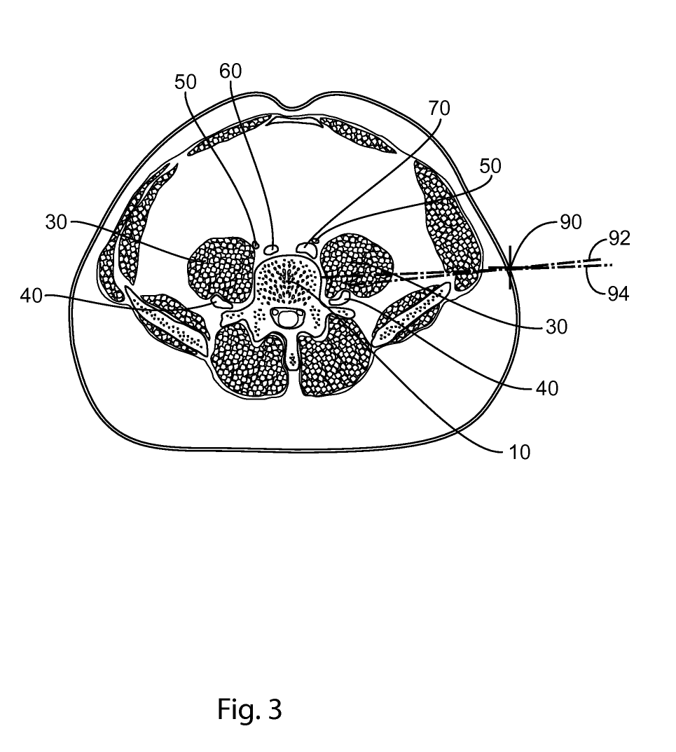

Referring to FIGS. 1-3, a vertebra 10 is shown with an adjacent intervertebral disc 20. A portion of a left psoas muscle 30 is shown laterally adjacent to the vertebra 10 and disc 20. The disc 20 may be accessed via a lateral approach through the psoas muscle 30. With specific reference to FIG. 3, the vertebra 10 and psoas muscle 30 are shown in a cross sectional view of a torso.

With continued reference to FIG. 3, the psoas muscle 30 incorporates nerves in its substance. In particular, a network of interlacing nerves, or lumbar plexus 40, is situated in the posterior part of the psoas muscle 30. The psoas muscle 30 is intimately associated with other anatomical structures, such as left and right ureters 50, abdominal aorta 60, inferior vena cava 70, and intestines 80. In the context of a lateral approach spinal procedure, these structures in and around the psoas muscle are clinically relevant because collateral damage to any one of these structures may cause undesirable effects, including pain, loss of function, bleeding, or death. In particular, collateral damage to nerves in and around the psoas muscle may cause significant and long lasting pain. Therefore, avoiding intra-operative insult to peri-psoas nerves may improve clinical outcomes, such as post-operative pain scores.

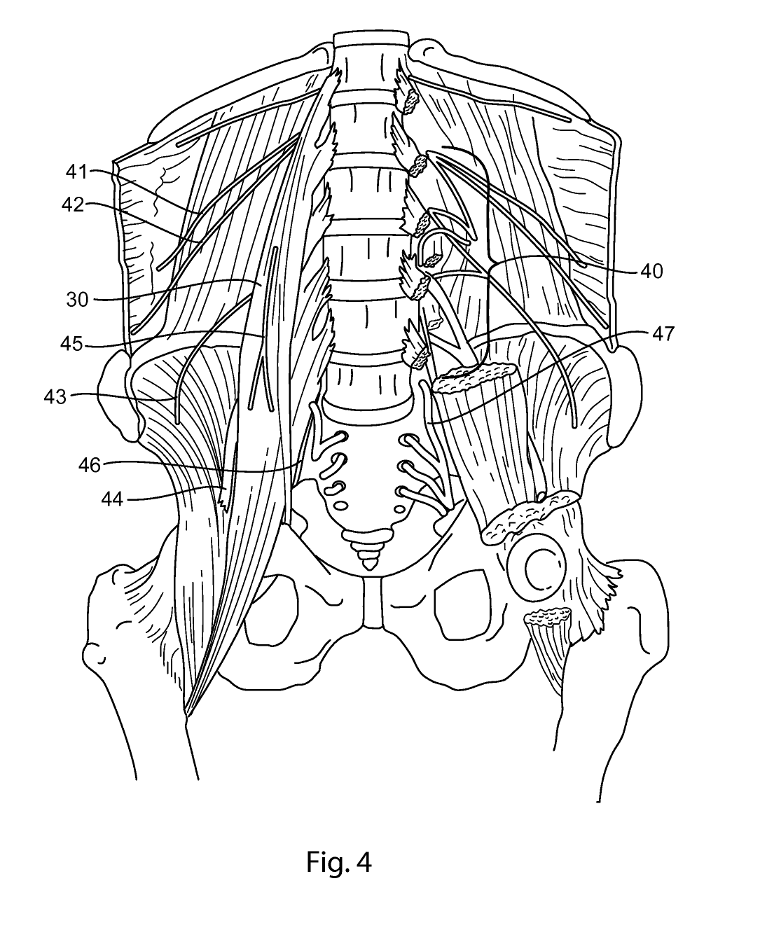

Referring to FIG. 4, a frontal view of the torso is shown. Details of the psoas muscle 30 and the lumbar plexus 40 are shown. Major nerves of the lumbar plexus 40 are shown. An iliohypogastric nerve 41, an ilioinguinal nerve 42, and a lateral femoral cutaneous nerve 43 emerge from the lateral border of the psoas muscle 30. A femoral nerve 44 emerges from a posterolateral aspect of the psoas muscle 30. A genitofemoral nerve 45 emerges anterior to the psoas muscle 30. An obturator nerve 46 and a lumbosacral trunk 47 emerge medial to the psoas muscle 30.

Returning to FIG. 1, an instrument 100 is shown extending mediolaterally through the psoas muscle 30 along a center longitudinal axis 101. Instrument 100 may have an imaging sensor 102 carried by a proximal working end 104 of a shaft 106. Sensor 102 may be permanently attached to shaft 106. In the embodiment of FIG. 1, the shaft 106 is straight and rigid. A distal end 108 of the shaft 106 may be physically coupled to an imaging console (not shown), for example, with a cable (not shown) to provide a communication link between sensor 102 and the console. Preferably, an outer diameter of the sensor 102 may be less than or equal to 6 mm. More preferably, an outer diameter of the instrument 100 may be less than or equal to 6 mm.

Sensor 102 may comprise an ultrasound transducer that may have a radial detection zone extending from a center point within the sensor 102. Preferably, the detection zone radius may be greater than or equal to 1 cm. The sensor 102 may be adjustable to alter the detection zone or to differentiate between various body structures. Sensor 102 may be a sterilizable reusable item.

The detection zone of sensor 102 may be planar such that it extends in two dimensions. A two-dimensional radial detection zone may be characterized as a circular segment, if it encompasses less than 360 degrees, or a circle, if it encompasses 360 degrees. The detection zone may lie in a plane that is perpendicular to axis 101, so that, for example, the detection zone extends around sensor 102, preferably in a full circle.

The imaging console (not shown) may comprise a sensor connection, such as a cable connection, a processor, software, a display, and a user interface. The imaging console may display an image acquired with the sensor 102 at a location within the psoas muscle 30. The imaging console may process data from the sensor to produce a refined image that accentuates differences between various body structures so that, for example, the image visually distinguishes nerves from all other body structures, generically described as flesh. The image may be monochromatic, such as grayscale. Preferably, the image may display different body structures in different hues so that, for example, blood vessels are red, nerves are yellow, muscle is green, and bone is blue. Additional detail about a particular body structure may be indicated in the image by varying the tint, shade, tone, or brightness of at least a portion of the structure. The imaging console may further comprise storage media so that an image may be saved for later retrieval.

In alternate embodiments of the invention, sensor 102 may be removably coupled to shaft 106. The alternate embodiment instrument 200 may have a shaft 206 that is bent or curved in at least one plane. The instruments 100, 200 may have a shaft that is rigid, flexible, or selectable between rigid and flexible. Distal ends 108, 208 may comprise a coupling 110, 210 or a handle (not shown).

In alternate embodiments, sensor 102 may be completely separate from instrument 100. Furthermore, sensor 102 may be carried by alternate instruments, such as a dilator, a hollow tube or cannula, a retractor, a speculum, or an implant inserter. An alternate embodiment contemplates a set of nesting cannulas and a solid dilator slidingly receivable within a smallest one of the cannulas, with a sensor carried on each component. Multiple sensors may be present on a single instrument.

The present invention may employ one or more of the following specialized ultrasound technologies: high-frequency, intra-vascular, two-dimensional, three-dimensional, or four-dimensional. Alternate imaging technology is also contemplated within the scope of the present invention. By way of non-limiting example, the invention could employ optical coherence tomography in place of, or in combination with, ultrasound imaging technology. One of ordinary skill in the art will recognize that additional alternate imaging technologies are also within the scope of the present invention to the extent that such imaging technologies are adaptable to the apparatus or methods set forth herein.

In alternate embodiments, the instrument 100 may wirelessly communicate with the imaging workstation. Sensor 102 may be a single use sterile packaged disposable item. The detection zone of sensor 102 may lie in a plane that is parallel to axis 101, so that, for example, the detection zone extends in front of sensor 102, and preferably in a circular segment evenly distributed on either side of axis 101. The detection zone of sensor 102 may be a three-dimensional spherical segment or full sphere. The image may be two-dimensional to correspond to a two-dimensional detection zone, or as a simplification of a three-dimensional detection zone. Alternatively, the image may be three-dimensional to correspond to a three-dimensional detection zone. The image may display different body structures in different tints, shades, tones, or brightnesses. The user may be able to zoom, pan, rotate, or otherwise manipulate the image or portions of the image through the user interface of the imaging console.

Referring to FIGS. 1-3, exemplary embodiments of methods according to the invention will be shown and described in detail in the context of a lateral approach spinal intervertebral fusion procedure. While these embodiments are shown and described in the context of a specific surgical application, one of ordinary skill in the art will appreciate that the present invention has utility in other surgical applications in various parts of the body.

The sensor 102 of instrument 100 may be placed against an entry point 90 on a lateral aspect of the body. The entry point may be prepared with a small incision so that the instrument slides easily through the skin. The sensor 102 may be inserted into the body so that the sensor 102 approaches a lateral aspect of the psoas muscle 30. As the instrument 100 is inserted through the body, the proximal end 104 and thus the sensor 102 may be maneuvered to selectively penetrate or slide past other body structures. Thus, the proximal end 104 and the sensor 102 establish a route 92 through the body, which the shaft 106 of the instrument 100 follows.

The sensor 102 may then be inserted into the psoas muscle 30 and advanced so that other sensor 102 approaches a lateral aspect of the intervertebral disc 20, thereby extending the route from the entry point to the disc 20. A passage is formed along the route 92 through the psoas muscle 30 as the sensor 102 is pushed through the psoas muscle 30.

As the sensor 102 is inserted into the body and advanced toward the disc 20, images may be acquired with the sensor 102. For example, an image may be acquired with the sensor 102 inserted halfway through the psoas muscle 30. Upon viewing the image, a surgeon may determine whether any nerve is present in the image. If a nerve, such as a nerve of the lumbar plexus 40, is present in the image, the surgeon may determine whether the nerve is an acceptable distance away from the sensor 102. If the nerve is spaced apart from the sensor 102, the surgeon may advance the sensor 102 closer to the disc 20 along the existing route 92. If the nerve is unacceptably close to the sensor 102, the surgeon may choose to move the sensor 102, and thus the proximal end 104 of the instrument 100, away from the nerve before advancing the sensor 102 closer to the disc 20 along a revised route 94. The surgeon may acquire and view images periodically or continuously in order to determine if any nerve is unacceptably close to the sensor 102.

Once the sensor 102 is at the lateral aspect of the disc 20, a surgical access channel may be formed by dilating the body structures surrounding the instrument 100. For example, a cannula or tube may be pushed over the instrument 100 to force apart surrounding tissues or body structures. Progressively larger cannulas may be added to create a surgical access channel of a desired size. All but the largest cannula may be removed such that the largest cannula holds the surgical access channel open. Alternatively, an adjustable retractor may be inserted around the instrument 100 and opened or spread in order to force apart surrounding tissues and hold the surgical access channel open. The retractor or any of the cannulas may carry an additional sensor so that images may be acquired and viewed during the process of creating the surgical access channel.

One or more surgical procedures may be performed on the disc 20 or in an intervertebral space created by removal of the disc 20. In this exemplary embodiment, disc 20 may be excised and the resulting intervertebral disc space may be filled with an intervertebral fusion prosthesis. The prosthesis or any surgical instrument used during the procedure may carry an additional sensor so that images may be acquired and viewed during or after the procedure.

An image enhancing medium may be introduced around the sensor 102 before or after acquiring an image with the sensor 102. The medium may acoustically couple the sensor 102 to surrounding body structures. For example, sterile saline or gel may be introduced around the sensor 102 to improve image quality through enhanced acoustic coupling. The image enhancing medium may be reserved for use only when image quality is suboptimal, or it may be introduced routinely, such as by flooding the vicinity with medium prior to introducing the sensor 102.

The revised route 94 may take advantage of the flexibility of flesh or the relative motility of organs or other body structures within the body so that it is not necessary to completely withdraw the sensor 102 in order to adopt the revised route 94. Rather, it may be possible to reorient sensor 102 onto the revised route 94 with minimal or partial withdrawal of sensor 102.

Although the exemplary embodiment stresses a route that avoids clinically relevant tissues, in an alternate embodiment, the surgical objective may be best served by a route defined by proximity to, or intersection with, a clinically relevant tissue, such as a nerve, blood vessel, or ligament.

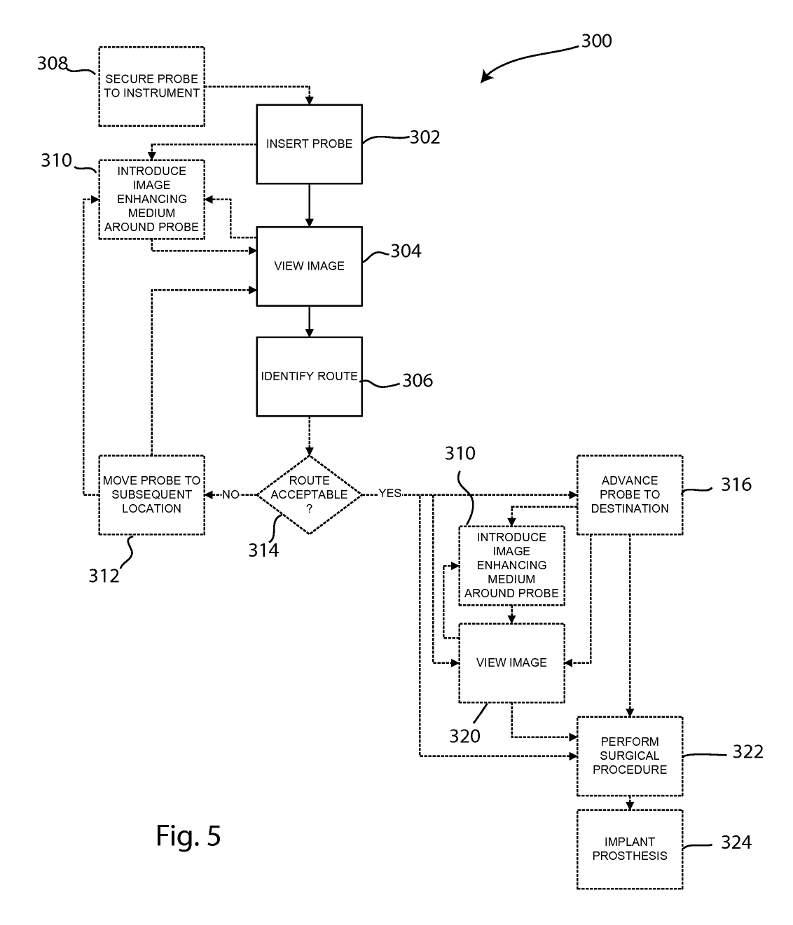

Referring to FIGS. 5-7, exemplary embodiments of methods according to the invention will be shown and described in the context of a lateral approach spinal intervertebral fusion procedure. One of ordinary skill in the art will appreciate that methods according to the present invention also have utility in other surgical applications in various parts of the body.

Referring to FIG. 5, a flow diagram shows a method 300 comprising three basic steps. A first step 302 may comprise inserting an imaging probe to a location within a body part. For example, step 302 may comprise inserting sensor 102 to a mid-substance location within the psoas muscle 30, adjacent to a portion of the lumbar plexus 40. A second step 304 may comprise viewing an image acquired with the probe at the location. For example, an image acquired with sensor 102 at the mid-substance location within the psoas muscle 30 may show no nerves proximate the sensor 102. A third step 306 may comprise identifying a route to a destination. For example, step 306 may comprise identifying a route to intervertebral disc 20. In this example, the image shows that the sensor 102 is on an acceptable route.

The method 300 may also comprise certain additional steps which may occur in relation to the steps 302, 304, 306.

A step 308 may comprise securing the probe at a proximal end of a selected one of a straight dilator shaft, a curved dilator shaft, a cannula, and a retractor, before performing step 302. Step 308 may be performed if, for example, sensor 102 is releasably securable to an instrument.

A step 310 may comprise introducing an image enhancing medium around the probe oat the location. As FIG. 5 shows, step 310 may occur at several points in method 300, since the purpose of step 310 is to enhance the image.

A step 316 may comprise advancing the probe to the destination along the route. For example, step 316 may comprise advancing sensor 102 along route 92 to disc 20. Step 316 may be performed when the route is acceptably oriented with regard to a clinically relevant tissue.

A step 312 may comprise moving the probe to a subsequent location within the body part. Step 312 may be performed when the route appears to be unacceptably oriented with regard to a clinically relevant tissue. For example, step 312 may comprise moving sensor 102 away from the portion of the lumbar plexus 40 so that sensor 102 lies along route 94 instead of route 92. Alternatively, step 312 may comprise moving sensor 102 toward a clinically relevant tissue, should it be desirable for the route to approach or intersect the clinically relevant tissue.

As shown in FIG. 5, step 312 may be followed by step 310 or step 304, so that a subsequent image acquired with the probe at the subsequent location may be viewed and a revised route to the destination may be identified. Thus steps 304 and 306 may be construed to pertain to the route or the revised route.

A decision step 314 may be inherent to the method 300. Decision step 314 may comprise deciding whether the route is acceptably or unacceptably oriented with respect to a clinically relevant tissue. Decision step 314 may be encountered repeatedly in method 300 until an acceptable route is identified.

A step 322 may comprise performing a surgical procedure at the destination, such as excising intervertebral disc 20. Step 322 may be followed by a step 324, comprising implanting a prosthesis, such as an intervertebral fusion cage. Alternatively, step 324 may be performed without performing step 322, in the situation where an implant requires no preparation of an implantation site.

A step 320 may comprise viewing a destination image acquired with the probe at the destination. As shown in FIG. 5, step 310 may occur before or after step 320 in order to enhance the image. Step 320 may advantageously confirm that the appropriate surgical destination has been reached, such as confirming the presence of disc degeneration prior to performing the surgical procedure.

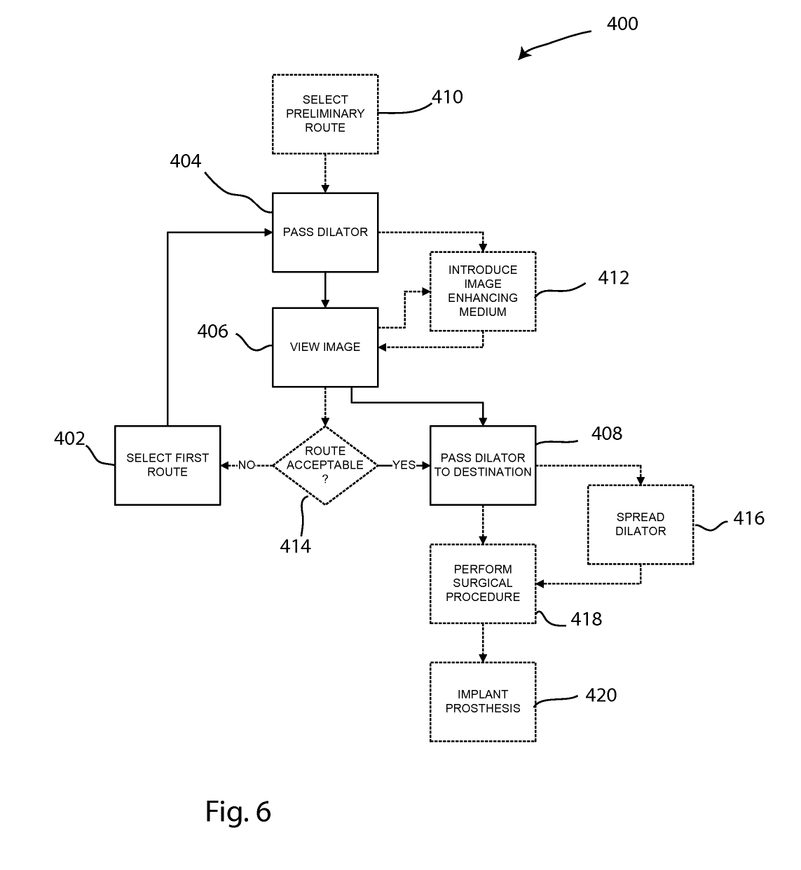

Referring to FIG. 6, a flow diagram shows a method 400 comprising four basic steps. A first step 402 may comprise selecting a first route from an entry point to the destination. For example, step 402 may comprise selecting route 94 from entry point 90 to intervertebral disc 20, as shown in FIG. 3. A second step 404 may comprise passing a first end of a dilator along the first route to a first location within the flesh. For example, step 404 may comprise passing proximal end 204 of instrument 100 along route 94 to a location spaced apart from a portion of the lumbar plexus 40. A third step 406 may comprise viewing an image acquired from an imaging probe at the first location, such as sensor 102 of instrument 100. A fourth step 408 may comprise passing the dilator along the first route from the first location to the destination.

The method 400 may also comprise certain additional steps which may occur in relation to the steps 402, 404, 406, 408.

A step 410 may comprise selecting a preliminary route from the entry point to the destination, prior to selecting the first route. For example, step 410 may comprise selecting route 92 from entry point 90 to intervertebral disc 20, as shown in FIG. 3.

As shown in FIG. 6, step 410 may be followed by step 404, so that the dilator may be passed along the preliminary route to a preliminary location within the flesh. Thus steps 404 and 406 may be construed to pertain to the first route or the preliminary route.

A step 412 may comprise introducing an image enhancing medium around the probe at the location. Step 412 may occur before or after step 406, since the purpose of step 412 is to enhance the image.

A decision step 414 may be inherent to the method 400. Decision step 414 may comprise deciding whether the route is acceptably or unacceptably oriented with respect to a clinically relevant tissue. Decision step 314 may be encountered repeatedly in method 400 until an acceptable route is identified.

A step 416 may comprise spreading the dilator to form a surgical access channel through the flesh, after passing the dilator along the first route from the first location to the destination. For example, step 416 may comprise spreading movable portions of a retractor from a more compact configuration to a more expanded configuration. Alternatively, step 416 may comprise enlarging a one-piece dilator.

A step 418 may comprise performing a surgical procedure at the destination. Step 418 may be followed by, or may incorporate, a step 420, comprising implanting a prosthesis. For example, steps 418 and 420 may comprise excising at least a portion of intervertebral disc 20 and inserting one or more intervertebral fusion spacers into the space previously occupied by disc 20.

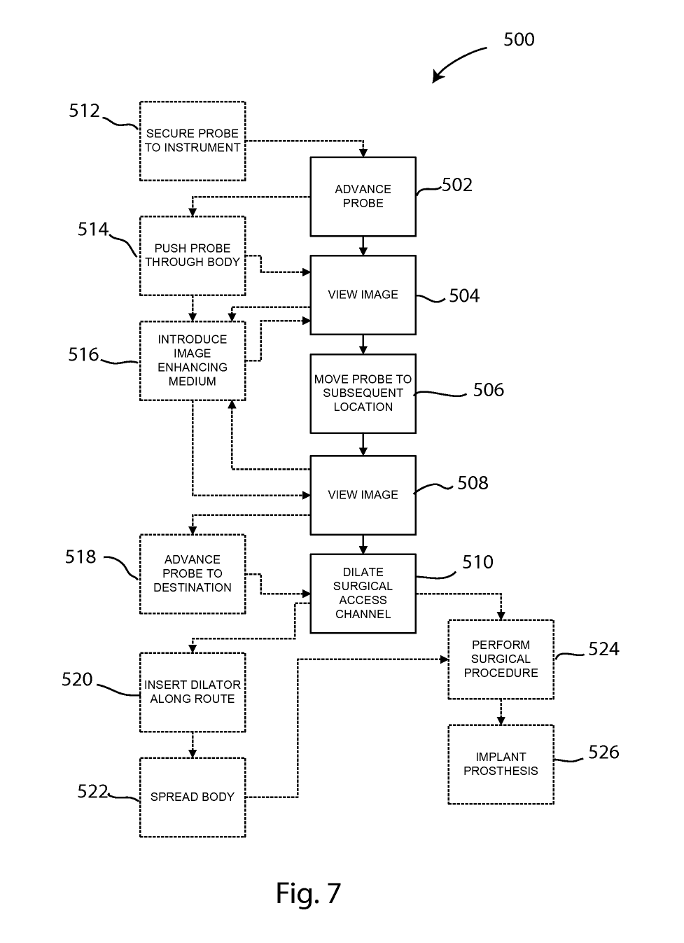

Referring to FIG. 7, a flow diagram shows a method 500 comprising five basic steps. A first step 502 may comprise advancing an imaging probe from an entry point to a first location within the body. For example, step 502 may comprise inserting sensor 102 to a mid-substance location within the psoas muscle 30, adjacent to a portion of the lumbar plexus 40. A second step 504 may comprise viewing a first image acquired from the probe at the first location. A third step 506 may comprise moving the probe to a subsequent location within the body. For example, step 506 may comprise moving sensor 102 away from the lumbar plexus 40. A fourth step 508 may comprise viewing a subsequent image acquired from the probe at the subsequent location. A fifth step 510 may comprise dilating a surgical access channel through the body following a route established by the probe through the entry point, the subsequent location, and the destination. For example, step 510 may comprise dilating a surgical access channel through the psoas muscle 30 and other body structures along route 94 (FIG. 3).

The method 500 may also comprise certain additional steps which may occur in relation to the steps 502, 504, 506, 508, 510.

A step 512 may comprise securing the probe at a proximal end of a selected one of a straight dilator shaft, a curved dilator shaft, a cannula, and a retractor blade, before advancing the probe to the first location.

A step 514 may comprise pushing the probe through the body to create the passage. For example, sensor 102 may be pushed through one or more muscle bellies, i.e., mid-substance, as it passes from the entry point to the first location within the body. FIGS. 1-2 show that sensor 102 has been pushed through the belly of the psoas muscle 30.

A step 516 may comprise introducing an image quality enhancing medium around the probe at the location. Steps 508 and 516 may also be performed in alternation until an acceptable image quality is achieved.

A step 518 may comprise advancing the probe to the destination along the route, before dilating the surgical access channel.

A step 520 may comprise inserting a dilator through the body following the route. For example, shaft 106 of instrument 100 may follow sensor 102 along the route. Alternatively, a separate dilator may be inserted along the route, either after removal of instrument 100 or with instrument 100 still in place along the route. The separate dilator may be structurally or functionally similar to a retractor or a speculum.

A step 522 may comprise spreading apart the body along the route to create the surgical access channel between the entry point and the destination. For example, step 522 may comprise passing a series of sequentially larger dilator cannulas over instrument 100 or a separate dilator in order to spread apart various body structures lying along the route. Alternatively, a collapsed retractor or speculum may be moved to an expanded configuration in order to spread apart a portion of the body in the vicinity of the route.

A step 524 may comprise performing a surgical procedure at the destination. Step 524 may be followed by, or may comprise, step 526, implanting a prosthesis.

While the foregoing disclosure sets forth exemplary embodiments of the present invention, one of ordinary skill in the art will appreciate that the apparatus and method of the present invention may be applicable throughout the body. By way of non-limiting example, the present invention may assist in navigating to surgical destinations near the cervical, brachial, lumbar, sacral, or myenteric plexuses, while avoiding the neural structures comprising these plexuses. The present invention contemplates an application in which posterior access to knee structures is facilitated by accurate visualization of circulatory and neural structures in the popliteal fossa.

1. A method of approaching a surgical destination through an adjacent body part, wherein a clinically relevant tissue is proximate the body part, comprising:
inserting an imaging probe to a location within the body part, wherein the probe creates a passage through the body part to the location;
viewing an image acquired with the probe at the location, wherein the image shows the body part adjacent to the probe, wherein the image visually distinguishes the tissue from the body part; and
identifying a route to the destination, wherein the route is defined by the tissue.

2. The method of claim 1, further comprising:
securing the probe at a proximal end of a selected one of a straight dilator shaft, a curved dilator shaft, a cannula, and a retractor, before inserting the probe to the location.

3. The method of claim 1, further comprising:
introducing an image enhancing medium around the probe at the location.

4. The method of claim 1, further comprising:
advancing the probe to the destination along the route.

5. The method of claim 1, further comprising:
moving the probe to a subsequent location within the body part;
viewing a subsequent image acquired with the probe at the subsequent location; and
identifying a revised route to the destination.

6. The method of claim 5, wherein the revised route is spaced apart from the tissue.

7. The method of claim 5, wherein the revised route intersects the tissue.

8. The method of claim 1, further comprising:
performing a surgical procedure at the destination.

9. The method of claim 8, wherein the surgical procedure comprises implanting a prosthesis.

10. The method of claim 1, further comprising:
viewing a destination image acquired with the probe at the destination.

11. The method of claim 1, wherein the destination is selected from the group consisting of an intervertebral disc and a vertebral body.

12. The method of claim 1, wherein the body part is a psoas muscle.

13. The method of claim 1, wherein the tissue is selected from the group consisting of nerves and blood vessels.

14. The method of claim 1, wherein the probe comprises an ultrasonic transducer.

15. The method of claim 1, wherein the image is a 360-degree circular view, wherein the circle lies in a plane, wherein the circle is centered on the probe, wherein the plane is perpendicular to a longitudinal axis of the probe.

16. The method of claim 1, wherein the image visually distinguishes the tissue from the body part by varying at least one of a hue, a tint, a shade, and a tone.

17. The method of claim 1, wherein the route is spaced apart from the tissue.

18. The method of claim 1, wherein the route intersects the tissue.

19. A method of approaching a surgical destination through adjacent flesh, wherein a clinically relevant tissue is proximate the flesh, comprising:
selecting a first route from an entry point to the destination;
passing a first end of a dilator along the first route to a first location within the flesh, wherein the dilator creates a passage through the flesh to the first location;
viewing an image acquired from an imaging probe at the first location, wherein the probe is carried by the first end of the dilator, wherein the image shows the flesh adjacent to the probe, wherein the image visually distinguishes the tissue from the flesh; and
passing the dilator along the first route from the first location to the destination.

20. The method of claim 19, further comprising:
selecting a preliminary route from the entry point to the destination, prior to selecting the first route;
passing the first end of the dilator along the preliminary route to a preliminary location within the flesh; and
viewing a preliminary image acquired from the probe at the preliminary location.

21. The method of claim 19, further comprising:
spreading the dilator to form a surgical access channel through the flesh, after passing the dilator along the first route from the first location to the destination.

22. The method of claim 19, further comprising:
introducing an image enhancing medium around the probe at the location.

23. The method of claim 19, further comprising:
performing a surgical procedure at the destination.

24. The method of claim 23, wherein the surgical procedure comprises implanting a prosthesis.

25. The method of claim 19, wherein the surgical destination is selected from the group consisting of an intervertebral disc and a vertebral body.

26. The method of claim 19, wherein the flesh is a psoas muscle.

27. The method of claim 19, wherein the tissue is selected from the group consisting of nerves and blood vessels.

28. The method of claim 19, wherein the dilator is a retractor.

29. The method of claim 19, wherein the probe comprises an ultrasonic transducer.

30. The method of claim 19, wherein the image is a 360-degree circular view, wherein the circle lies in a plane, wherein the circle is centered on the probe, wherein the plane is perpendicular to a longitudinal axis of the probe.

31. The method of claim 19, wherein the image visually distinguishes the tissue from the flesh by varying at least one of a hue, a tint, a shade, and a tone.

32. The method of claim 19, wherein the first route is spaced apart from the tissue.

33. The method of claim 19, wherein the first route intersects the tissue.

34. A method of approaching a surgical destination within a body of a vertebrate animal, wherein a clinically relevant tissue is positioned within the body, comprising:
advancing an imaging probe from an entry point to a first location within the body;
viewing a first image acquired from the probe at the first location, wherein the first image shows a portion of the body proximate the probe, wherein the first image visually distinguishes the tissue from the body;
moving the probe to a subsequent location within the body;
viewing a subsequent image acquired from the probe at the subsequent location, wherein the subsequent image shows a portion of the body proximate the probe, wherein the subsequent image shows that the tissue is in a desired orientation relative to the subsequent location; and
dilating a surgical access channel through the body following a route established by the probe through the entry point, the subsequent location, and the destination.

35. The method of claim 34, further comprising:
securing the probe at a proximal end of a selected one of a straight dilator shaft, a curved dilator shaft, a cannula, and a retractor blade, before advancing the probe to the first location.

36. The method of claim 34, wherein advancing the probe comprises pushing the probe through the body to create the passage.

37. The method of claim 34, further comprising:
introducing an image quality enhancing medium around the probe at the location, before viewing one of the first and subsequent images.

38. The method of claim 34, further comprising:
advancing the probe to the destination along the route, before dilating the surgical access channel.

39. The method of claim 34, wherein dilating the surgical access channel comprises:
inserting a dilator through the body following the route; and
spreading apart the body along the route to create the surgical access channel between the entry point and the destination.

40. The method of claim 39, wherein the dilator is a retractor.

41. The method of claim 34, further comprising:
performing a surgical procedure at the destination.

42. The method of claim 41, wherein the surgical procedure comprises implanting a prosthesis.

43. The method of claim 34, wherein the surgical destination is selected from the group consisting of an intervertebral disc and a vertebral body.

44. The method of claim 34, wherein the body comprises a psoas muscle.

45. The method of claim 34, wherein the tissue is selected from the group consisting of nerves and blood vessels.

46. The method of claim 34, wherein the probe comprises an ultrasonic transducer.

47. The method of claim 34, wherein the image is a 360-degree circular view, wherein the circle lies in a plane, wherein the circle is centered on the probe e, wherein the plane is perpendicular to a longitudinal axis of the probe.

48. The method of claim 34, wherein the image visually distinguishes the tissue from the body by varying at least one of a hue, a tint, a shade, and a tone.

49. The method of claim 34, wherein the route is spaced apart from the tissue.

50. The method of claim 34, wherein the route intersects the tissue.

51. A system for approaching a surgical destination through an adjacent body part, wherein a clinically relevant tissue is proximate the body part, comprising:
an instrument having a working end and a second end opposite the working end;
an imaging probe carried by the working end of the instrument; and
an imaging console in communication with the probe, wherein, when the working end of the instrument is placed at a location within the body part, the console displays an image acquired from the probe, wherein the tissue is visually distinct from the flesh in the image.

52. The system of claim 51, wherein the surgical destination is selected from the group consisting of an intervertebral disc and a vertebral body.

53. The system of claim 51, wherein the body part comprises a psoas muscle.

54. The system of claim 51, wherein the tissue is selected from the group consisting of nerves and blood vessels.

55. The system of claim 51, wherein the instrument is a dilator.

56. The system of claim 55, further comprising a set of nesting cannulas, wherein the dilator is slidingly received within a smallest one of the cannulas.

57. The system of claim 51, wherein the instrument is a cannula.

58. The system of claim 51, wherein the instrument is a retractor.

59. The system of claim 51, wherein the probe comprises an ultrasonic transducer.

60. The system of claim 51, wherein the console is in wireless communication with the probe.

61. The system of claim 51, wherein the image is a 360-degree circular view, wherein the circle lies in a plane, wherein the circle is centered on the probe, wherein the plane is perpendicular to a longitudinal axis of the probe.

62. The system of claim 51, wherein the image visually distinguishes the tissue from the body by varying at least one of a hue, a tint, a shade, a tone, and a brightness.
